(19) United States
(12) Patent Application Publication
(10) Pub. No.: US 2012/0010630 A1
(43) Pub. Date: Jan. 12, 2012

(54) System and Method for Hair Removal

(76) Inventors: Angela Ahmadi Miller, Dallas, TX (US); Azam Anwar, Dallas, TX (US); Christopher Michael Boykin, Athens, TX (US)

(21) Appl. No.: 12/832,804

(22) Filed: Jul. 8, 2010

(51) Int. Cl. A61B 17/30

(52) U.S. Cl. 606/133

(57) ABSTRACT

A method in one example embodiment includes providing first and second devices, each of which includes an actuating body coupled to a pair of bifurcated arms with distal tips. The method also includes moving at least one of the devices to create tension in a filament secured to each of the distal tips and then forming a twist in the filament between the distal tips of the first device and the distal tips of the second device. The method further includes placing a first portion of the filament around hairs to be removed from a body. An opening force is applied to the actuating body of the second device to move the bifurcated arms from a first position to a second position having a widened distance between the distal tips, such that the twist travels along the filament toward the first device catching and removing hairs from the body.

### TECHNICAL FIELD

This disclosure relates in general to the field of esthetics and, more particularly, to a system and method for hair removal.

### BACKGROUND

The field of esthetics has seen tremendous growth over the last several years. Esthetics involves beautifying the skin in a variety of ways, including makeup application, facial treatments, skin and body treatments, and chemical treatments. Esthetics also includes hair removal treatments, both on the face and on other parts of the body. Cultures typically have societal norms regarding an “ideal” amount of body hair on males and females. Unwanted hair that does not conform to the arbitrary “ideal” amount of hair may cause social discomfort and, possibly, lower self-esteem. In Western culture, the trend toward hairlessness in women developed and became entrenched as a societal norm in the 1900s. Today, many women routinely apply hair removal techniques to various parts of their bodies, including parts of their faces such as, for example, around eyebrows, around lips, on necks, and any other areas in which unwanted hair appears. Many men also use hair removal techniques, such as shaving facial and neck hair.

Most hair removal techniques have become commonplace, with some performed at home, some performed by medical professionals, and others performed in salons by estheticians or other beauty specialists. Many of the current hair removal procedures (e.g., waxing, plucking, laser, chemical, etc.) suffer from significant drawbacks, including pain to the treated area and resulting skin problems. The demand for these procedures, however, continues to rise. Accordingly, alternative techniques that minimize pain and skin problems would be beneficial to users who perform these techniques on themselves and also operators who perform these techniques on clients.

### BRIEF DESCRIPTION OF THE DRAWINGS

FIG. 1 is a side, top, and front perspective view of one embodiment of a threading device in a contracted position in accordance with the present disclosure;

FIG. 2 is a top plan view of the threading device of FIG. 1 in a contracted position;

FIG. 3 is a front elevation view of the threading device of FIG. 1 in an contracted position;

FIG. 4 is a rear elevation view of the threading device of FIG. 1 in an contracted position;

FIG. 5 is a right side elevation view of the threading device of FIG. 1 in a contracted position;

FIG. 6 is a left side elevation view of the threading device of FIG. 1 in an expanded position;

FIG. 7 is cross-sectional view of the threading device taken along 7-7 of FIG. 5;

FIG. 8A is a perspective view of a threading system with right and left hands in phantom holding threading devices prepared with an untwisted filament in one example operation in accordance with the present disclosure;

FIG. 8B is a side elevation view of the threading system of FIG. 8A showing the filament twisted, with one threading device in a contracted position and the other threading device in an expanded position;

FIG. 8C is a side elevation view of the threading system of FIG. 8A shown with the twisted filament placed around a line of hairs on a facial area of human skin to be removed;

FIG. 8D is a side elevation view of the threading system of FIG. 8A showing the line of hairs being removed upon actuation of the threading system;

FIG. 9 is a top, side, and front perspective view of a second embodiment of a threading device in accordance with the present disclosure;

FIG. 10 is a top plan view of the threading device of FIG. 9;

FIG. 11 is a front elevation view of the threading device of FIG. 9 in an expanded position;

FIG. 12 is a rear elevation view of the threading device of FIG. 9 in an expanded position;

FIG. 13 is a right side elevation view of the threading device of FIG. 9 in a contracted position;

FIG. 14 is a left side elevation view of the threading device of FIG. 9 in an expanded position;

FIG. 15 is cross-sectional view taken along 15-15 of FIG. 14;

FIG. 16 is a perspective view of one of a pair of bifurcated arms removed from the threading device of FIG. 9;

FIG. 17 is a top, side, and front perspective view of the threading device of FIG. 9 including an alternative embodiment of a pair of bifurcated arms in a contracted position in accordance with the present disclosure;

FIG. 18 is a perspective view of one of the pair of bifurcated arms removed from the threading device of FIG. 17;

FIG. 19 is a top, side, and front perspective view of another embodiment of a threading device in accordance with the present disclosure;

FIG. 20 is a right side elevation view of the embodiment of the threading device of FIG. 19 in an expanded position; and

FIG. 21 is a right side elevation view of the threading device of FIG. 19 in a contracted position.

### DETAILED DESCRIPTION OF EXAMPLE EMBODIMENTS

### Overview

A method is provided in one example embodiment including providing first and second devices, each of which includes an actuating body coupled to a pair of bifurcated arms with distal tips. A filament is secured to each of the distal tips. The method includes moving at least one of the first and second devices to create tension in the filament. The method also includes forming a twist in the filament between the distal tips of the first pair of bifurcated arms and the distal tips of the second pair of bifurcated arms. The twist separates a first portion of the filament adjacent the first device and a second portion of the filament adjacent the second device. The method further includes placing the first portion of the filament around hairs to be removed from a body. In addition, an opening force is applied to the actuating body of the second device to move the bifurcated arms of the second device from a first position to a second position. The second position has a widened distance between the distal tips causing the twist to travel along the filament toward the first device catching and removing hairs from the body.

### Example Embodiments

To provide a more complete understanding of the present disclosure and features and advantages thereof, reference is made to the accompanying figures in which like reference numerals reference like parts. Where two devices of the same embodiment are shown in the same FIGURE, the additional device is distinguished with the addition of apostrophes for ease of reference in the description. Where alternative embodiments are shown, common elements are similarly numbered and not separately described, with the addition of apostrophes to distinguish the embodiments.

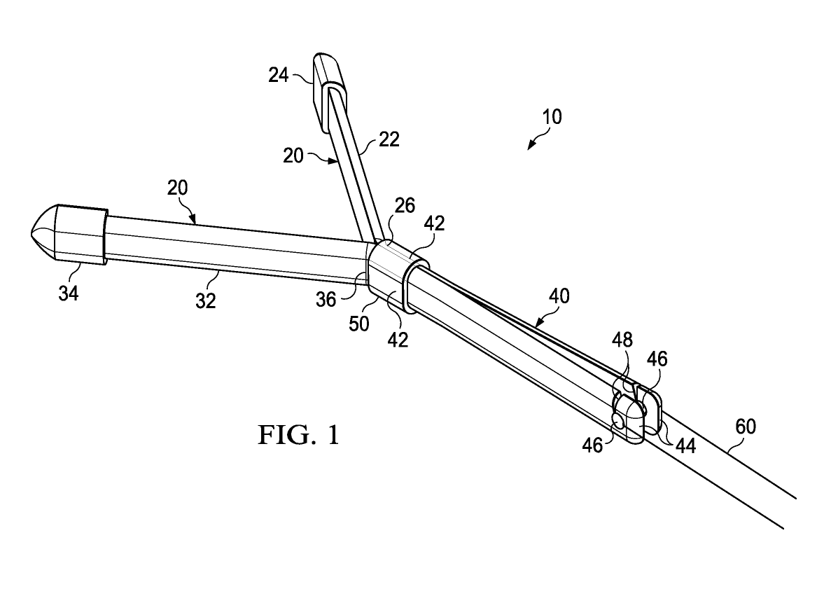

With initial reference to FIG. 1, one embodiment of a threading device 10 that may be used in a hair threading procedure is shown. Threading device 10 may include an actuating body 20 and a pair of bifurcated arms 40 pivotally joined by an elastic sleeve 50. Actuating body 20 may include first and second members 22 and 32 each having respective rearward ends 24 and 34 and front ends 26 and 36. Rearward ends 24 and 34 may have caps 29 to aid human hands in securely gripping first and second members 22 and 32. Each bifurcated arm 40 may have a proximal end 42 attached to one of first and second members 22 and 32 at front ends 26 and 36, respectively. Each bifurcated arm 40 may also have a distal tip 44 with an eyelet 46 formed therein and a slit 48 in communication with eyelet 46. A filament 60 may be secured to distal tips 44 of bifurcated arms 40.

In example embodiments, threading device 10 may be used in tandem with a second threading device having the same or similar design to perform a threading procedure to remove hairs from a body area. Threading devices may be held by left and right hands and pulled away from each other to create tension in filament 60. The hands may rotate one or both threading devices to form a twist in filament 60 between distal tips 44 of threading device 10 and distal tips of the second threading device. After filament 60 is placed over one or more hairs to be removed from a body, human hands may apply forces to first and second members of the actuating bodies to expand and contract the bifurcated arms in order to cooperatively move the twist between the threading devices. The hairs may then be caught by the movement of the twist and pulled out of the body. The forces may be selectively applied to the actuating bodies to move the twist a particular length along filament 60 in order to quickly extract a desired amount of hairs from a line of hairs on the body. In addition, threading devices as shown and described in this disclosure may protect persons who perform the threading technique from rubbing and cuts on their hands, necks, and gums, which can be caused by customary threading techniques.

For purposes of teaching and discussion, it is important to understand the environment in which threading device 10 may be used. The following foundational information may be viewed as a basis from which the present disclosure may be properly explained. Such information is offered earnestly, for purposes of explanation only and, accordingly, should not be construed in any way to limit the broad scope of the present disclosure and its potential applications and embodiments.

Although numerous hair removal techniques are available (e.g., waxing, plucking, laser hair removal, chemicals, shaving), each can be problematic for recipients of such techniques. For example, waxing can be painful and cause skin problems such as redness, swelling, tenderness and scabbing. Waxing is also believed to accelerate wrinkling, particularly in the delicate tissue around the eyes. Moreover, medical doctors advise patients taking certain medications to avoid waxing as it could permanently damage their skin, which may have a heightened sensitivity due to the medication. Laser hair removal is an alternative, sometimes permanent, hair removal technique. It can be cost prohibitive, however, for many individuals. In addition, when laser hair removal is used around the eyebrows, it could prevent the ability of an individual to ever change the shape of their eyebrows (e.g., adjust the arch, grow thicker eyebrows, etc.). Shaving is another technique, but hair is cut at the surface of the skin and, therefore, shaving must be performed more often than other techniques. Many people also experience “razor burn” or skin irritation using a razor, in addition to accidental cuts by the blade. Shaving is also not recommended around the eye area because of the risk of such cuts. Plucking is another alternative, but is time consuming because typically, only one hair at a time may be removed. Consequently, the pain is prolonged because each hair must be individually removed, and the skin area is often red and swollen afterwards. Finally, hair removal chemicals can be damaging to sensitive skin, can leave skin red and “burned”, and are often not safe for use on the face and in particular, the sensitive eye area.

Threading is an alternative hair removal technique used in various parts of the world that can minimize pain and resulting skin problems to the treated body area. Typically, threading is inexpensive if performed in a salon, and can be quickly completed by a competent operator. It is a precise technique in which a line of hairs or individual hairs may be quickly removed from the hair follicle. Accordingly, the hair removal may last several weeks. If performed properly, threading may also be less painful than other hair removal techniques such as plucking individual hairs, waxing, or the like.

Typically, threading is used to remove unwanted hair from facial areas, such as eyebrows, unibrows, and upper lips. Threading, however, can also be used for hair removal on any other accessible parts of the body. A filament such as, for example, cotton thread, is commonly held by a person's fingers, mouth, and/or neck to perform threading. Particular techniques of threading may vary depending on whether threading is performed by a person to remove hair from another person's body, or to remove hair from one's own body.

When threading is performed on oneself, a filament, such as cotton thread, may be tied to form a closed loop of any desired size. For some individuals, a desirable thread length is 20-26 cm long. Fingers from each hand are inserted through the closed loop and moved in opposing directions to create tension in the loop. Some persons use a thumb and three fingers to control the thread movement. The person may rotate one or both hands to create a twist in the loop, such that the twist connects two closed loop portions of the thread and when the hands are moved in opposing directions to create tension in the thread, two generally triangular shapes may be formed in the thread with the twist disposed therebetween. Typically, the hands are rotated to make from two to six twists in the thread.

The person prepares to remove unwanted hair by determining a direction of growth of the hair to be removed and which hand to place next to the hair to be removed. The person then spreads the fingers of this placing hand wide to force the twist to travel along the thread loop toward the opposite actuating hand, creating an enlarged closed loop area adjacent the placing hand. Then the person places the thread such that the enlarged closed loop area surrounds the hair to be removed and the twist is placed in the direction of the hair growth adjacent to the first hair to be removed. The person then spreads the fingers of the opposite actuating hand, forcing the twist to travel along the thread loop toward the placing hand such that the enlarged closed loop area is reduced in size and any hairs within the path of the twist as it travels are caught and pulled from the body.

When an operator performs threading on a client, the procedure may vary. For example, instead of using one hand as the actuating hand, the operator may use a combination of mouth and hand or neck and hand. For example, the operator may use one hand as the placing hand with a thumb and one or more fingers inserted in the loop, and then the operator may take part of the thread and loop it around one or more teeth and use the other actuating hand to pull the thread wide to move the twist along a path of hairs to be removed. In this case, the thread may or may not form a continuous closed loop as one end may be in the mouth and the other end may be in the actuating hand. Alternatively, some operators may put a thread loop around their neck rather than in their mouth.

Although threading offers many benefits over other hair removal procedures, traditional threading methods often cause pain to the fingers, hands, neck, or gums around which the thread is wrapped. As the thread rubs against the skin, it can create raw areas of the skin and even cuts in the skin or gums. While the person receiving the treatment may get the benefits of threading hair removal, the person performing the threading procedure may experience skin and/or gum problems and pain resulting from the use of the thread. Additionally, placing a portion of the thread in an operator's mouth while the thread is used to perform a procedure on another person can be unsanitary.

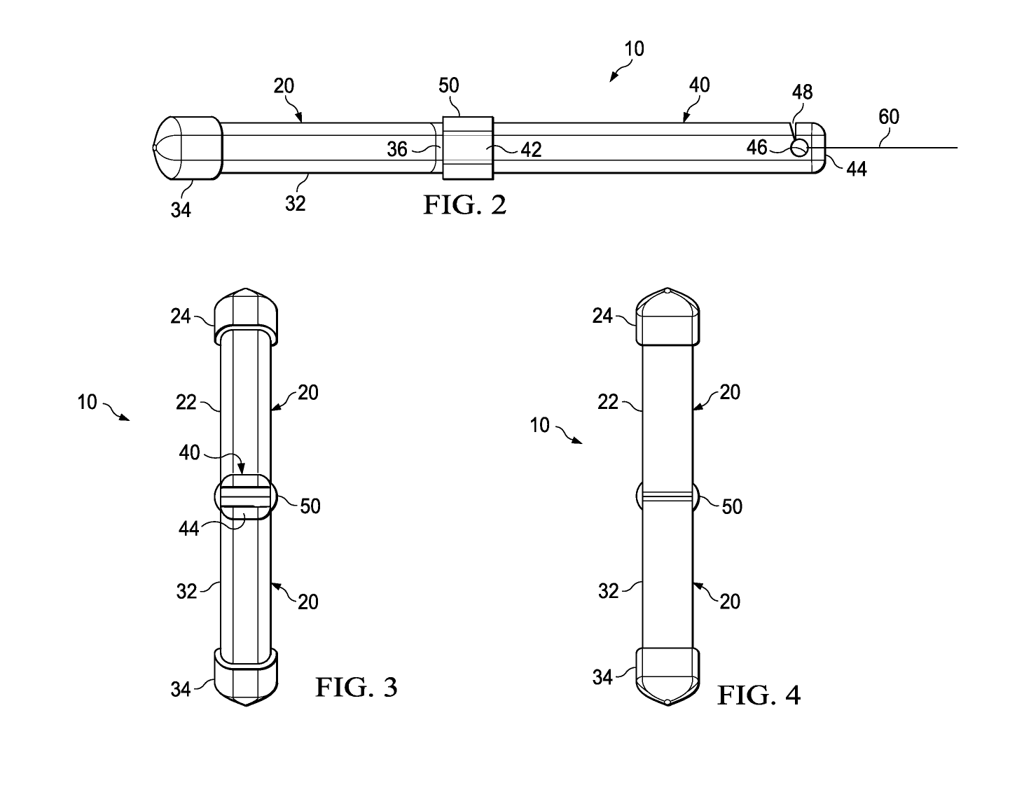

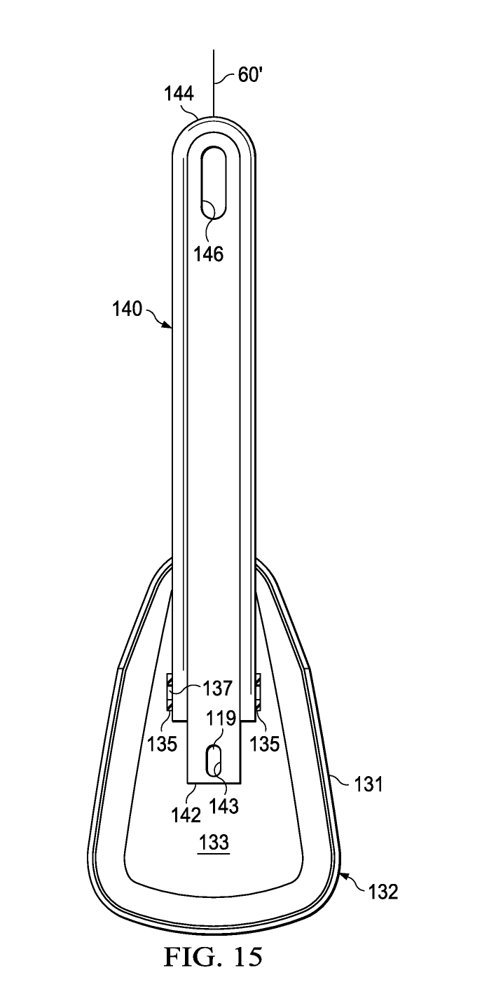

Threading device embodiments as shown in FIGS. 1-21 overcome many of these problems and provide a solution for individuals who perform threading on themselves and for operators who perform threading on clients. Its unique concept and embodiments allow individuals and operators to perform threading without any of their body parts (e.g., fingers, thumbs, necks, gums) touching the thread during a procedure. Thus, use of a threading device, as shown and described in this disclosure, prevents cutting and rubbing on fingers, thumbs, necks, or gums. Furthermore, use of such threading devices enables operators to perform threading without putting the thread in their mouths, thereby providing a more sanitary procedure.

Turning to the particular features of the example embodiment of threading device 10 shown in FIGS. 1-7, first and second members 22 and 32 of actuating body 20 may be configured in generally rectangular, elongated shapes and biased by elastic sleeve 50 such that a longitudinal axis of first member 22 forms an angle with a longitudinal axis of second member 32. In one example embodiment, the angle may be less than 90 degrees when threading device 10 is at rest (i.e., when no external forces are being applied to actuating body 20). However, the angle may be any suitable size that permits a user to comfortably grip first and second members 22 and 32 and apply a force to cause first rearward ends 24 and 34 to pivot toward each other and actuate bifurcated arms 40. Bifurcated arms 40 may also be configured in substantially rectangular, elongated shapes. In one embodiment, however, bifurcated arms 40 may be generally contiguous when threading device 10 is at rest and may extend outwardly from front ends 26 and 36 of first and second members 22 and 32, respectively, such that each bifurcated arm 40 has a longitudinal axis forming an obtuse angle with the longitudinal axis of the first or second member 22 or 32 to which it is attached.

Eyelets 46 formed in distal tips 44 of each bifurcated arm 40 may be adapted to slidably receive filament 60. Filament 60 may be, for example, cotton thread, all purpose thread, polyester thread, or any other thin string having sufficient frictional forces when twisted together to accomplish hair removal using a threading procedure. For example, string with serrated or looped fibers that permit a twist to move back and forth over a looped portion of the string and catch and pull any hairs in the path the twist travels may be used. Each distal tip 44 may also have an edge with slit 48 formed therein and being in communication with eyelets 46, for easily loading filament 60 into eyelets 46. This may be particularly useful as a user can tie a filament into a loop and then load the loop onto threading device 10 through slits 48.

In one example embodiment, first members 22 and 32 and their corresponding bifurcated arms 40 may be held together by a fulcrum in the form of some type of an elastic element, such as elastic sleeve 50. Elastic sleeve 50 may be positioned to surround proximal ends 42 of bifurcated arms 40, adjacent front ends 26 and 36 of first and second members 22 and 32. Elastic sleeve 50 may facilitate pivotal movement of first and second members 22 and 32 relative to each other, so that as rearward ends 24 and 34 pivot toward each other, distal tips 44 of bifurcated arms 40 pivot away from each other and as rearward ends 24 and 34 pivot away from each other, distal tips 44 of bifurcated arms 40 pivot toward each other.

Actuating body 20 and bifurcated arms 40 may be formed of any suitable material (e.g., high density polyethelyne (HDPE), polyvinyl chloride (PVC), sheet metal, etc.) or any combination of such materials. In one embodiment, each of first and second members 22 and 32 may be molded together with one of bifurcated arms 40 to form a single molded piece, such that each proximal end 42 of bifurcated arms 40 is integrally molded with one of front ends 26 and 36 of first and second members 22 and 32. The two single molded pieces may then be pivotally joined by elastic sleeve 50. Elastic sleeve 50 may be made of any suitable elastomer (e.g., natural rubber, synthetic rubber, etc.). The broad scope of this disclosure allows any suitable alternative attachment mechanism (e.g., bolts, screws, glue, snap fit, press fit, etc.) to be used to connect proximal ends 42 of bifurcated arms 40 to first and second members 22 and 32. The individual components, including single molded pieces and elastic sleeve 50, may be formed through any acceptable method such as, for example, injection molding, or laser, mechanical, or chemical milling, or any type of metal fabrication process if metal is used.

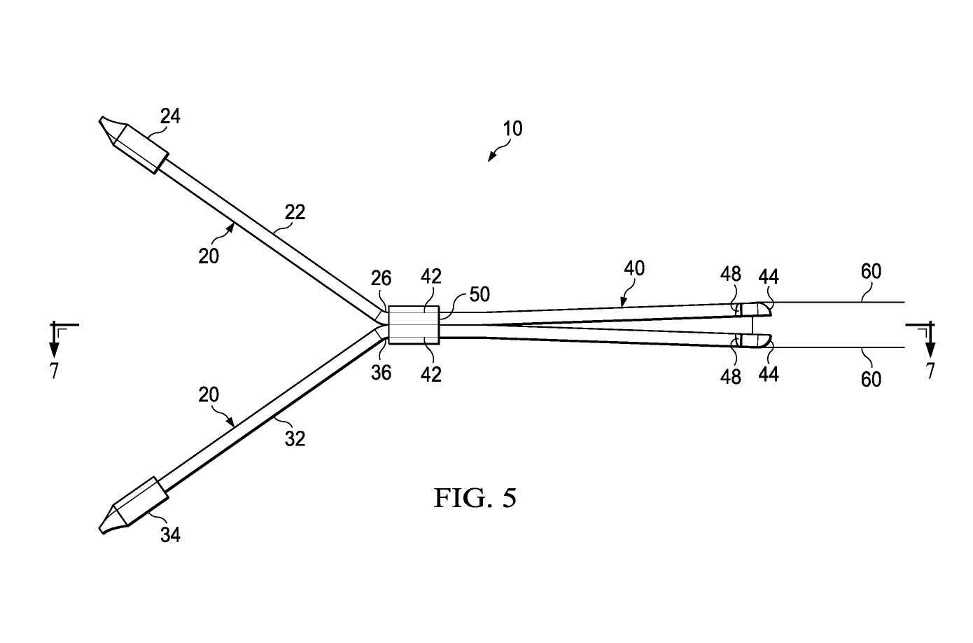

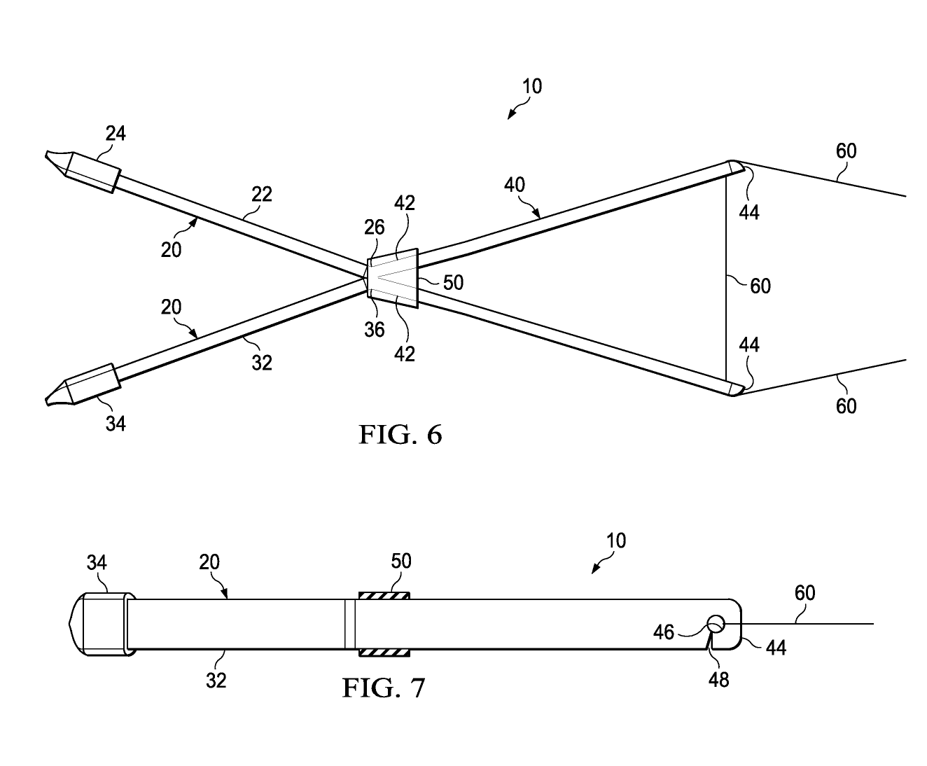

With reference to FIGS. 5 and 6, bifurcated arms 40 of threading device 10 are shown in a first position and a second position, respectively. In FIG. 5, bifurcated arms 40 are generally contiguous in the first position and are contracted such that a minimum narrowed distance is defined between distal tips 44. In one embodiment, bifurcated arms 40 are biased to the first position by elastic sleeve 50. In FIG. 6, bifurcated arms 40 are expanded such that a maximum widened distance is defined between distal tips 44. In one embodiment, the second position is achieved when a contracting force is applied to first and second members 22 and 32 of actuating body 20. Threading device 10 may be configured using different dimensions and angles to accommodate varying minimum and maximum distances between distal tips 44 when bifurcated arms 40 are moved between first and second positions. In an example embodiment, the minimum narrowed distance may be from 0 to 2 inches in length, and the maximum widened distance may be from 2 to 6 inches in length. Distal tips 44 may be configured to approach closure, or to achieve closure (i.e., 0 inches) for a minimum narrowed distance, as this allows a twist formed in filament 60 to be moved closer to distal tips 44.

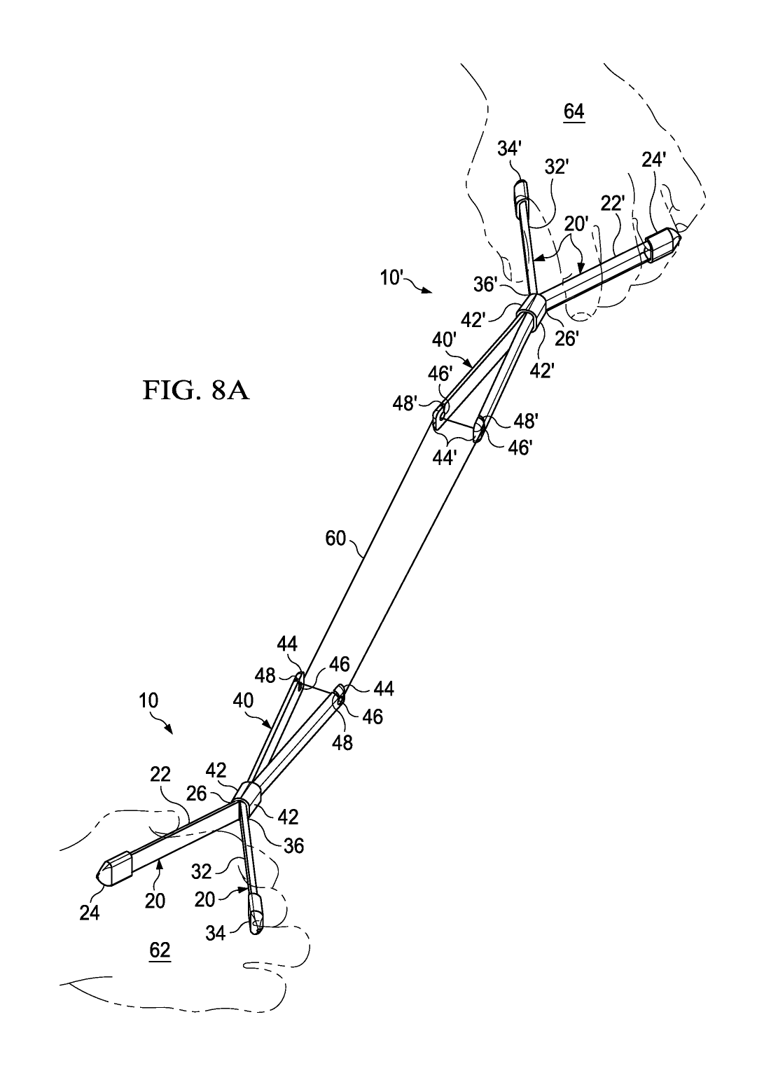

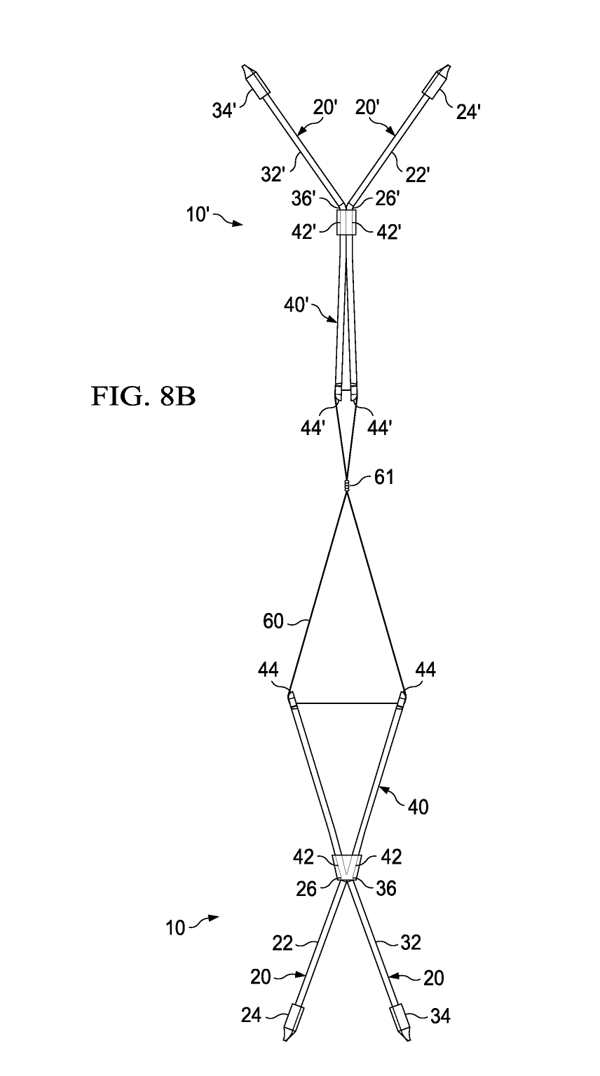

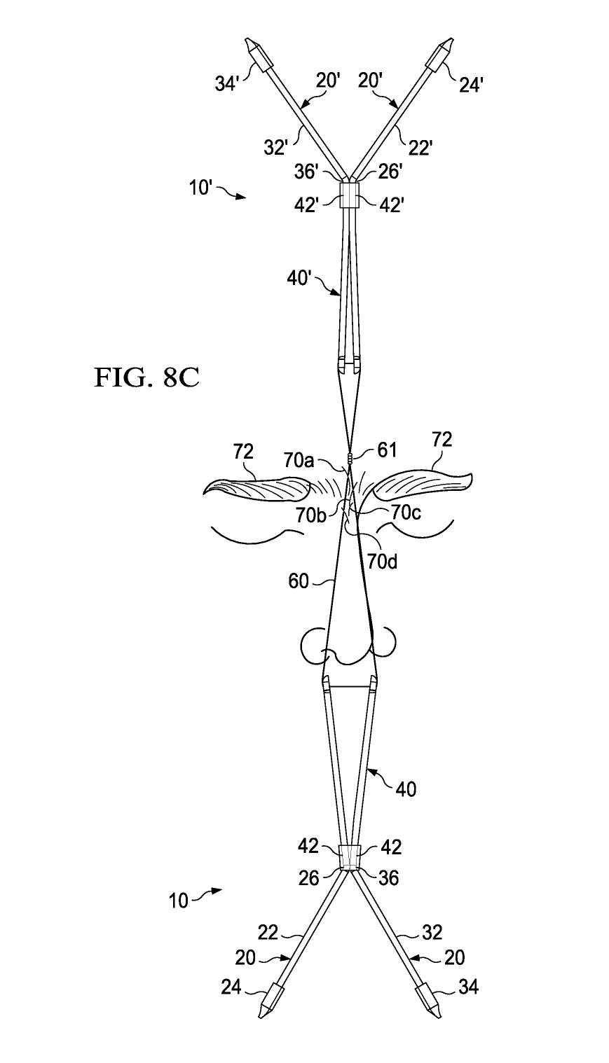

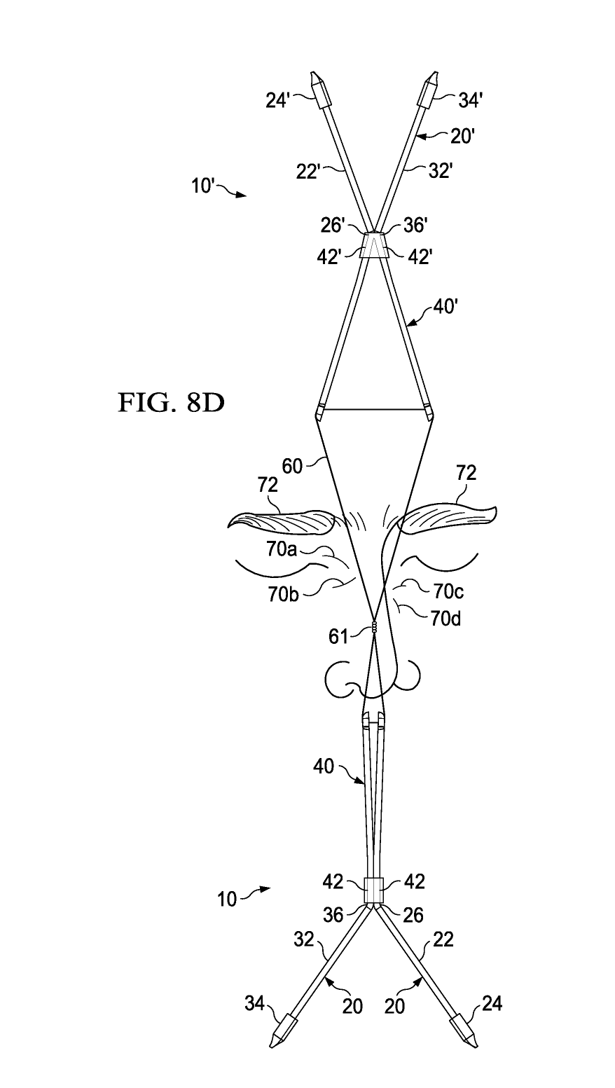

FIGS. 8A through 8D illustrate an example threading procedure using two identical threading devices 10 and 10′. For ease of reference, human hands 62 and 64 are shown only in FIG. 8A. However, it will be apparent the tensioning and contracting forces shown in FIGS. 8B, 8C, and 8D may also be applied by human hands. With reference to FIG. 8A, a schematic view of threading devices 10 and 10′ held by right and left hands 62 and 64, respectively, prepared with a looped filament 60 is shown. Looped filament 60 may be inserted through both eyelets 46 and through both eyelets 46′. A knot may be tied in looped filament 60 such that threading devices 10 and 10′ are linked together. Right and left threading devices 10 and 10′ may be pulled in opposing directions to create tension in filament 60. When this tension is created, filament 60 is formed into a substantially rectangular shape with eyelets 46 and eyelets 46′ establishing corners of the rectangular shape. One or both threading devices may be twisted around a longitudinal axis of devices 10 and 10′ in order to form twist 61 in filament 60 between distal tips 44 of threading device 10 and distal tips 44′ of threading device 10′. Alternatively, filament 60 may be tied in a knot before being loaded onto threading devices 10 and 10′. In this alternative procedure, filament 60 may be loaded through slits 48 and 48′ to be received in eyelets 44 and 44′. It will be apparent that twist 61 could also be formed before loading filament 60 onto threading devices 10 and 10′.

With reference to FIG. 8B, the rotation has been performed by one or both threading devices 10 and 10′ and a twist 61 has been created in filament 60. In one example scenario, twist 61 may be formed in looped filament 60 after three rotations. A contracting force is applied to first and second members 22 and 32 of actuating body 20 whereby bifurcated arms 40 are expanded to the second position with a maximum widened distance between distal tips 44. An external force has not been applied to threading device 10′ and, therefore, bifurcated arms 40′ remain in the first position with a minimum narrowed distance between distal tips 44′. The expansion of bifurcated arms 40 into the second position causes twist 61 to move toward threading device 10′, thus preparing the system for removing hair from a body. An enlarged triangular shape may be formed in filament 60 between twist 61 and distal tips 44 of threading device 10.

With reference to FIG. 8C, filament 60 is shown being placed around a line of hairs 70a, 70b, 70c, and 70d to be removed on a body. In this example, hairs 70 are situated in a unibrow area of an individual's forehead, between eyebrows 72, and are growing in an upward direction. The enlarged triangular shape created in filament 60 is placed to surround the line of hairs 70 with twist 61 in the upward growth direction of hairs 70 and adjacent to a first hair 70a to be removed. The user may release some of the force on right hand threading device 10, allowing bifurcated arms 40 to retract somewhat so that twist 61 will be able to travel along filament 60 when bifurcated arms 40′ of threading device 10′ are expanded.

With reference to FIG. 8D, a contracting force is applied to first and second members 22′ and 32′ of actuating body 20′ so that bifurcated arms 40′ expand into the second position. As bifurcated arms 40′ are expanding, twist 61 travels along filament 60 and catches hairs 70a, 70b, 70c, and 70d, respectively, and pulls them from hair follicles under the surface of the skin. The process may be repeated until all unwanted hair is removed. In addition, when a line of hairs is growing in an opposite direction, the operation and placement of threading devices 10 and 10′ may simply be reversed to remove such hairs. This process effectively removes a line of hairs precisely and quickly, and threading devices 10 and 10′ prevent the user's hands, gums, and neck from receiving cuts and/or burns from filament 60 rubbing against the user's skin or gums during the threading procedure. In addition, if threading devices 10 and 10′ are used by an operator on a client, threading devices 10 and 10′ make a sanitary procedure possible as the need for putting filament 60 in the operator's mouth is eliminated.

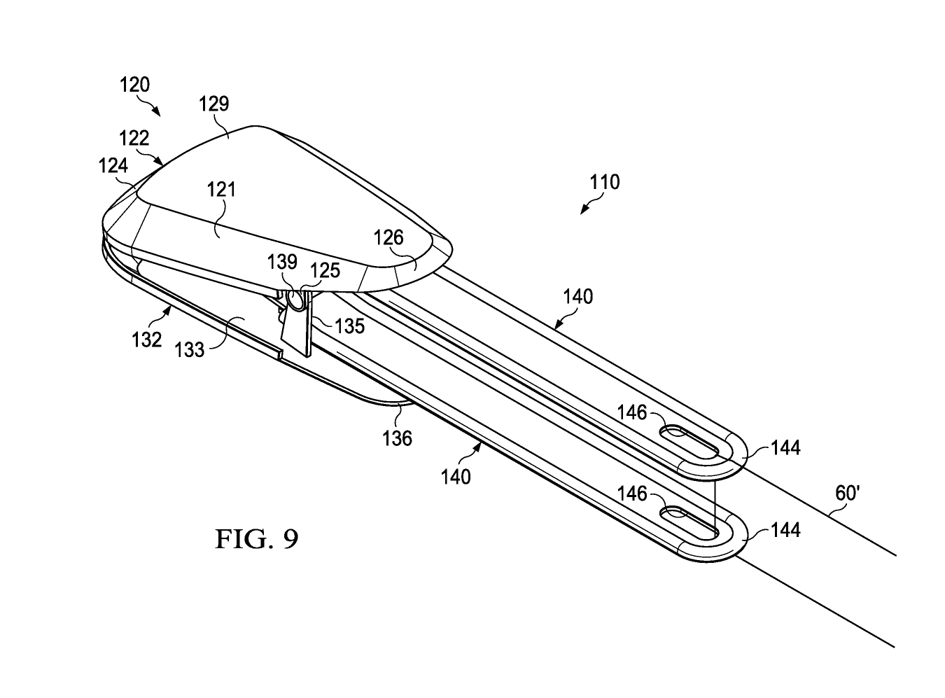

FIGS. 9-15 illustrate a second embodiment of a threading device 110 that may be used in a hair threading procedure. With initial reference to FIG. 9, threading device 110 may include an actuating body 120 having a first member 122 and an opposing second member 132. First member 122 may have an outer surface 121, an inner surface 123, a rearward end 124, a front end 126, and two projections 125 extending outwardly from inner surface 123 and positioned substantially perpendicular to a longitudinal axis of first member 122. Second member 132 may also have an outer surface 131, an inner surface 133, a rearward end 134, a front end 136, and two projections 135 extending outwardly from inner surface 133 and positioned substantially perpendicular to a longitudinal axis of first member 122. First and second members 122 and 132 may also have snap protrusions 119 extending outwardly from inner surfaces 123 and 133, respectively, which may be laterally centered on first and second members 122 and 132, respectively. Projections 125 and 135 may have holes 127 and 137 formed therein and adapted to slidably receive pin 139 when holes 127 and 137 are in axial alignment. Projections 125 and 135 may be positioned between front ends 126 and 136 and rearward ends 124 and 134, respectively, such that pin 139 provides pivotal movement to first and second members 122 and 132. First and second members 122 and 132 of actuating body 120 may be provided with gripping pads 129 to allow a user to more easily grip actuating body 120 and retain the hold without slippage. Gripping pads 129 may, in one embodiment, be made of any suitable material that provides some friction and comfort to human skin (e.g., rubber, foam, soft plastic, etc.).

A pair of bifurcated arms 140 may extend outwardly from front ends 126 and 136 of first and second members 122 and 132, respectively. Each of the pair of bifurcated arms 140 may have a proximal end 142 attached to one of first and second members 122 and 132 and a distal tip 144 having an eyelet 146 formed therein. Eyelets 146 may be adapted to slidably receive a filament 60′. Proximal ends 142 of bifurcated arms 140 may each have, in one embodiment, an opening 143 formed therein, which can be removably snapped onto snap protrusions 119. It will be apparent, however, that proximal ends 142 may be connected to first and second members 122 and 132 in any suitable way (e.g., bolts, screws, glue, snap fit, press fit, integral plastic molding, etc.). In one embodiment, each bifurcated arm 140 may be configured to extend outwardly along a longitudinal axis of its respective first or second member 122 or 132. Alternatively, bifurcated arms 140 could be angled away from each other to provide a greater maximum distance between distal tips 144, when performing a threading procedure.

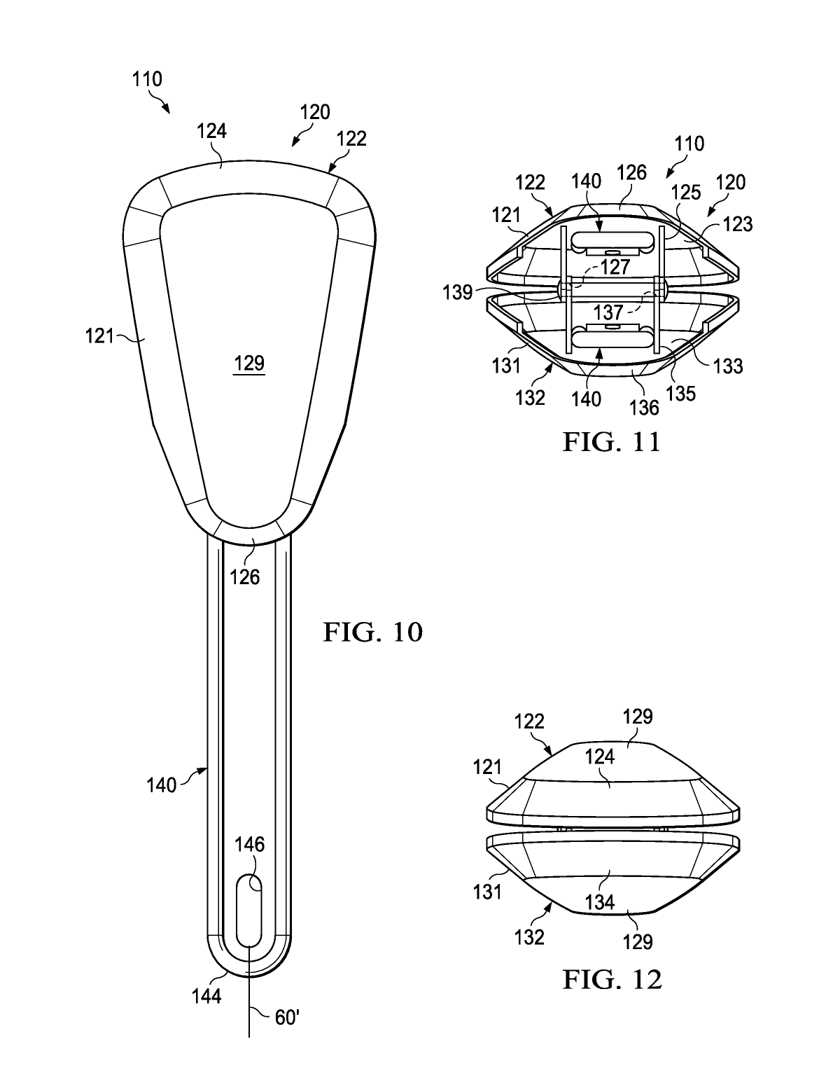

In FIG. 10, a top plan view of threading device 110 is shown. In one example embodiment, first and second members may be generally triangular in shape with rounded edges and sized to fit in a human hand. Bifurcated arms 140 may be configured into any suitable shapes including, for example, long, flat, substantially rectangular shapes with rounded distal tips 144 as shown in the embodiment in FIG. 10.

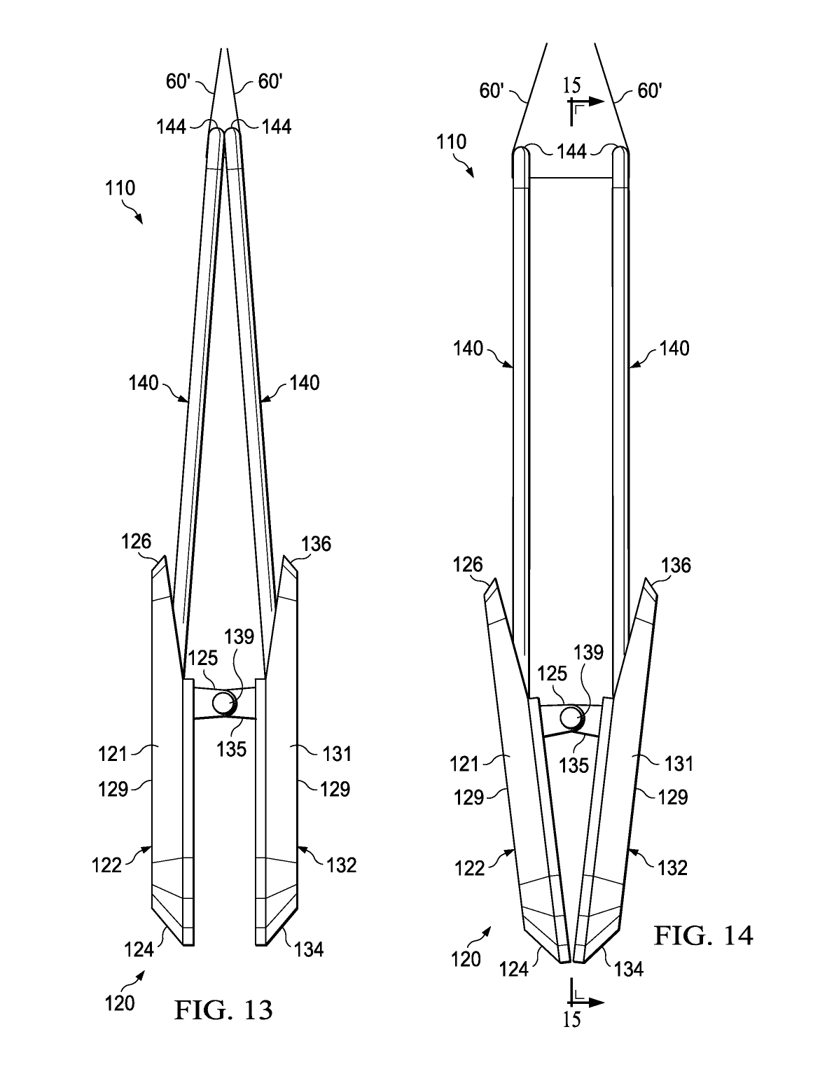

FIGS. 13 and 14 illustrate a right side elevation view with bifurcated arms 140 in a first position and a left side elevation view with bifurcated arms 140 in a second position, respectively. In FIG. 13, first and second members 122 and 132 are fully pivoted forward such that distal tips 144 have converged into a first position with no spacing therebetween. A contracting force may be applied to front ends 126 and 136 in order to pivot first and second members 122 and 132 forward. In FIG. 14, first and second members 122 and 132 are fully pivoted backward so that rearward ends 124 and 134 have converged and bifurcated arms 140 have expanded to the second position having a maximum widened distance between distal tips 144 of bifurcated arms 140. A contracting force may be applied to rearward ends 124 and 134 in order to pivot first and second members 122 and 132 backward. In one embodiment, the pivoting occurs about pivot pin 139. Alternatively, a biasing mechanism such as, for example, a coil spring or clothespin spring, may be used to bias front and second members 122 and 132 in either a first position, fully pivoted forward, or in a second position, fully pivoted backward. In such an embodiment, moving bifurcated arms 140 between first and second positions may only require a contracting force may to be applied to overcome the resistance of the biasing mechanism in the biased first or second position to the opposing first or second position. Threading device 110 may be configured using different dimensions to accommodate varying maximum distances between distal tips 144 when bifurcated arms 140 are expanded from the first position to the second position. In an example embodiment, the maximum widened distance may be from 1.5 to 3 inches in length.

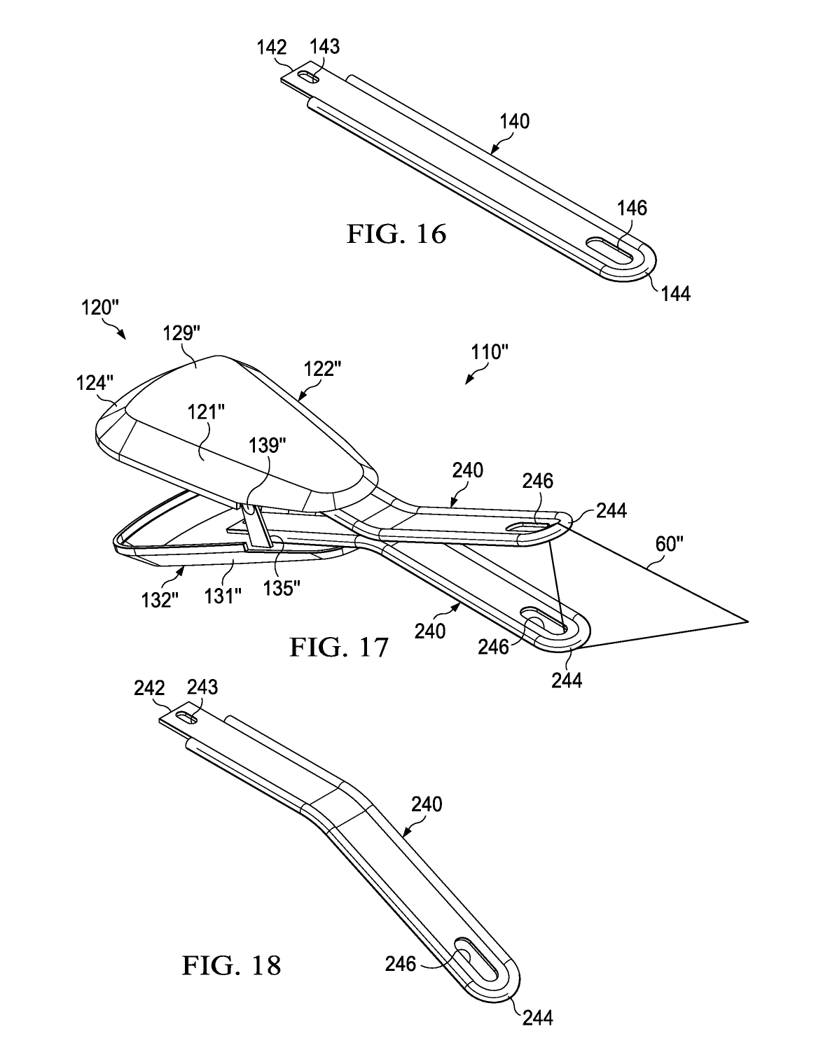

With reference to FIG. 16, one of the pair of bifurcated arms 140 is shown removed from actuating body 110. In one embodiment, bifurcated arm 140 may be a substantially straight, flat, rectangular shape with distal tip 144 having a rounded shape and proximal end 142 being narrowed. Proximal end 142 may also have an opening 143 formed therein for snap fitting onto protrusion 119 of first or second member 122 or 132.

Actuating body 120 and bifurcated arms 140 may be formed of any suitable material (e.g., high density polyethelyne (HDPE), polyvinyl chloride (PVC), sheet metal, etc.) or any combination of such materials. In one embodiment, each first and second member 122 and 132 may be molded and joined with pin 139, and may include protrusions 119 adapted to receive proximal ends 142 of bifurcated arms 140. The broad scope of this disclosure, however, allows for any suitable attachment mechanism (e.g., bolts, screws, glue, snap fit, press fit, etc.) to be used to connect proximal ends 142 of bifurcated arms 140 to first and second members 122 and 132. Alternatively, each bifurcated arm 140 may be integrally molded with one of first and second members 122 and 132. The individual components of actuating body 120 and bifurcated arms 140 may be formed through any acceptable method such as, for example, injection molding, or laser, mechanical, or chemical milling, or any suitable type of metal fabrication process if metal is used.

FIGS. 17 and 18 illustrate another embodiment of a threading device 110″ in which a pair of bifurcated arms 240 being used with threading device 110″ are alternatively configured. Proximal ends 242 of bifurcated arms 240 may be attached in any suitable way to first and second members 122″ and 132″, as previously discussed with reference to threading device 110. In one example embodiment, bifurcated arms 240 may angle outwardly from front ends 126″ and 136″ away from each other, such that they are disposed in divergent planes, creating greater distances between distal tips 244 when bifurcated arms 240 are in a first position and in a second position. Such a configuration facilitates a greater expansion between distal tips 244 when threading device 110″ is pivoted to expand bifurcated arms 240. Thus, a greater maximum widened distance may be defined between distal tips 244. This greater expansion may compress a twist in a filament 60″ and allow the twist to travel further along filament 60″ toward an opposite threading device when first and second members 122″ and 132″ of threading device 110″ are pivoted backward.

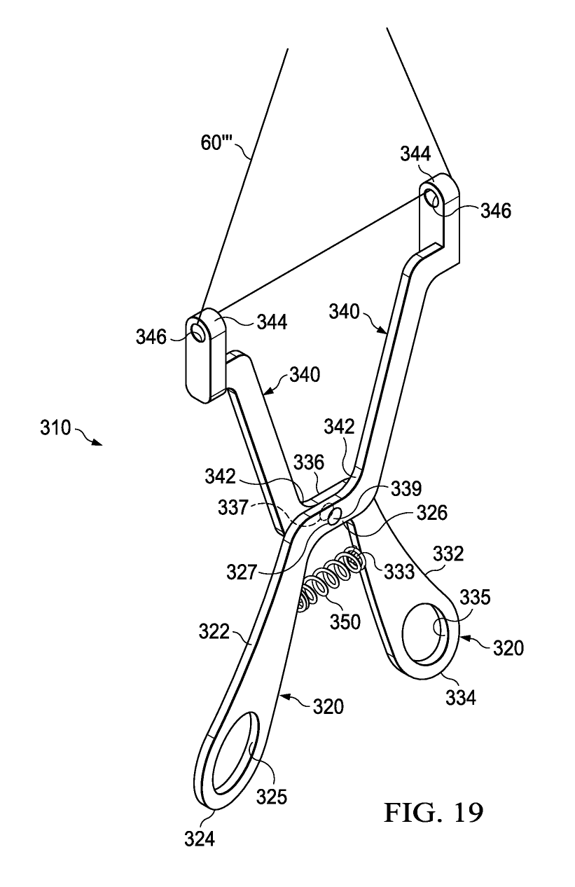

FIGS. 19-21 illustrate a third embodiment of a threading device 310, which may be used in a threading procedure. Threading device 310 may include an actuating body 320 and a pair of bifurcated arms 340 pivotally joined by a pin 339 and a spring 350. Actuating body 320 may include first and second members 322 and 332 each having respective rearward ends 324 and 334, front ends 326 and 336, and finger openings 325 and 335 defined in respective first and second members 322 and 332 adjacent rearward ends 324 and 334 for receiving human fingers to control threading device 310. First and second members 322 and 332 may be elongated and biased by spring 350 into an expanded position such that a longitudinal axis of first member 322 forms an angle with a longitudinal axis of second member 332 when threading device 310 is at rest (i.e., when no external forces are being applied to actuating body 320). First and second members 322 and 332 may be configured to define any suitable angle that permits a user to comfortably grip first and second members 322 and 332 and apply a force to cause rearward ends 324 and 334 to pivot toward each other such that distal tips 344 may be sufficiently contracted and expanded. Front end 336 of second member 332 may be configured to angle laterally toward first member 322 and to have a hole 337 defined therein. Front end 326 of first member 322 may be configured to angle laterally toward second member 332 and to also have a hole 327 defined therein, such that holes 327 and 337 may be axially aligned to slidably receive pin 339. Lips 323 and 333 on opposing edges of first and second members 322 and 332 may be positioned adjacent front ends 326 and 336 for receiving a spring 350.

Bifurcated arms 340 may extend outwardly from front ends 326 and 336 of first and second members 322 and 332, respectively. Each bifurcated arm 340 may have a proximal end 342 integrally formed with one of front ends 326 and 336 and a distal tip 344 with an eyelet 346 formed therein and adapted to slidably receive a filament 60′″. Bifurcated arms 340 may be configured in substantially flat, elongated shapes. In one embodiment, however, each distal tip 344 may be laterally offset from a longitudinal axis of its respective bifurcated arm 340, and oriented such that eyelets 346 are generally in axial alignment. Bifurcated arm 340 connected to first member 322 may be disposed in a same plane with first member 322 and generally oriented along an axis parallel to a longitudinal axis of first member 322 as front end 326 may laterally offset bifurcated arm 340 from first member 322. Similarly, bifurcated arm 340 connected to second member 332 may be disposed in a same plane with second member 332 and generally oriented along an axis parallel to a longitudinal axis of second member 332 as front end 336 may laterally offset bifurcated arm 340 from second member 332.

Actuating body 320 and bifurcated arms 340 may be formed of any suitable material (e.g., high density polyethelyne (HDPE), polyvinyl chloride (PVC), metal, etc.) or any combination of such materials. In one embodiment, each of first and second members 322 and 332 may be integrally formed or molded together with one of bifurcated arms 340 to form a single component. The two single components may be pivotally joined by pin 339 and biased in an expanded position by spring 350. The broad scope of this disclosure, however, permits bifurcated arms 340 to be formed as separate components and connected to first and second members 322 and 332 using any suitable attachment mechanism (e.g., bolts, screws, glue, snap fit, press fit, etc.). The individual components, including single molded pieces, may be formed through any acceptable method such as, for example, injection molding, or laser, mechanical, or chemical milling, or any suitable type of metal fabrication process for metal.

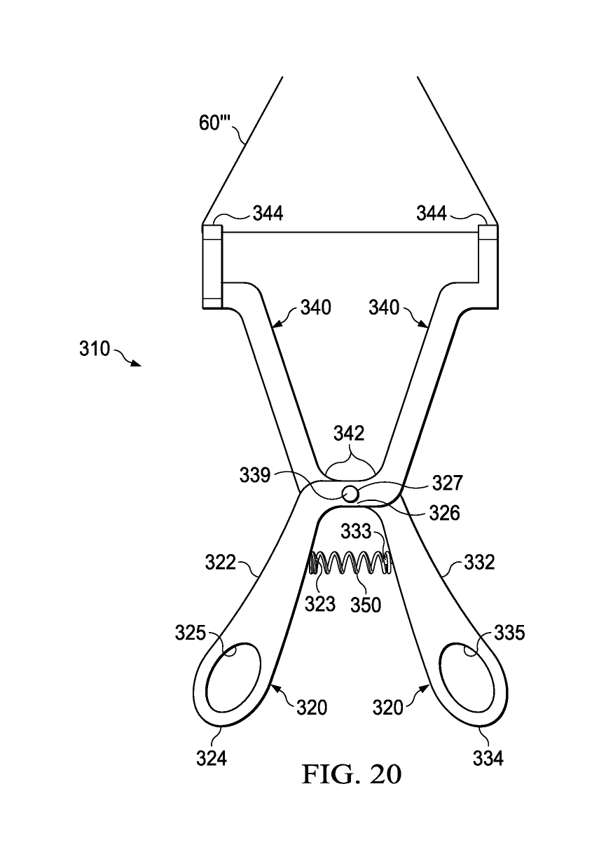

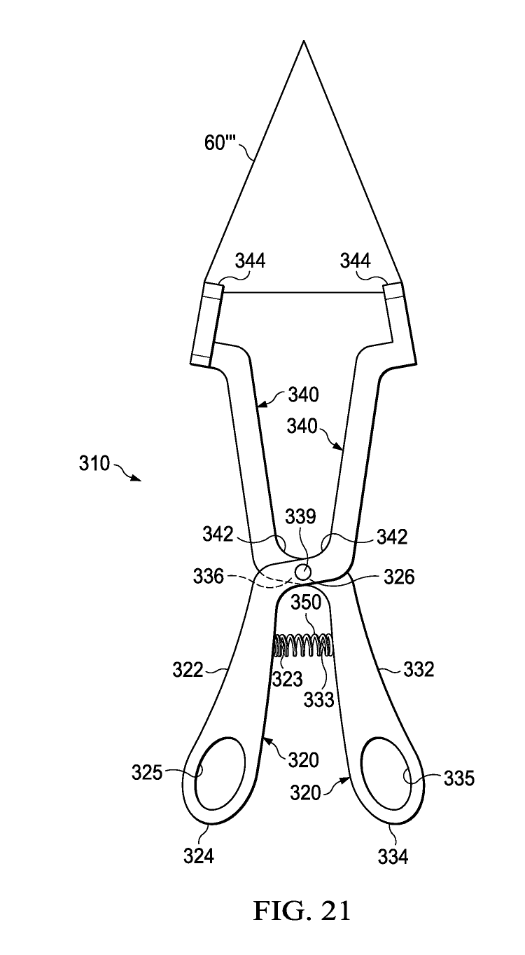

With reference to FIGS. 20 and 21, bifurcated arms 340 of threading device 310 are shown in a second position and a first position, respectively. In FIG. 20, bifurcated arms 340 are expanded to the second position and a maximum widened distance is defined between distal tips 344. In one embodiment, first and second members 322 and 332 are biased to this second position by spring 350 when no external forces are being applied to threading device 310. In FIG. 21, bifurcated arms 340 are contracted, being generally contiguous and having a minimum narrowed distance defined between distal tips 344. In one embodiment, the first position is achieved when a contracting force is applied to first and second members 322 and 332 of actuating body 320 to overcome the resistance of spring 350. Threading device 310 may be configured using different dimensions and angles to accommodate varying minimum and maximum distances between distal tips 344 when bifurcated arms 340 are moved between first and second positions. In an example embodiment, the minimum narrowed distance may be from 0 to 2 inches in length, and the maximum widened distance may be from 2 to 6 inches in length. Distal tips 344 may be configured to approach closure, or to achieve closure (i.e., 0 inches) for a minimum narrowed distance, as this allows a twist formed in filament 60′″ to be moved closer to distal tips 344 to facilitate placing the twist near hair to be removed from a body.

In operation, threading device 310 as shown in FIGS. 19-21 will require a user or operator to apply a contracting force to first and second members 322 and 332 to contract or close distal tips 344 and then release the force to allow spring 350 to expand first and second members 322 and 332, thereby expanding distal tips 344 of bifurcated arms 340. Thus, contracting forces applied to threading device 310 would be applied during different operational steps than the contracting forces applied with respect to threading device 10 described with reference to example threading procedure of FIGS. 8B-8D.

Threading device 310 may also be alternatively designed without a biasing mechanism, such as spring 350. In such an alternative embodiment, a user or operator would have to apply an expanding force to first and second members 322 and 332 to expand or open distal tips 344. In addition, the user or operator would also have to apply a contracting force to contract or close distal tips 344. In this alternative embodiment, however, the contracting force would not have to overcome the resistance of a biasing mechanism such as spring 350.

It is important to note that the stages and steps in the preceding FIGURES illustrate only some of the possible situations that may be executed by, or within, the designs of the present disclosure. Some of these stages and/or steps may be deleted or removed where appropriate, or these stages and/or steps may be modified or changed considerably without departing from the scope of the present disclosure. In addition, the timing of these operations may be altered considerably. For example, while two threading devices (e.g., threading devices 10 and 10′) may be used to perform a threading procedure, an operator may choose to use only one threading device and continue to hold a portion of filament 60 using, for example, their hand, mouth, and/or neck. In addition, as previously noted herein, an operator holding a threading device in each hand may use either hand to place filament 60 around hairs to be removed and the opposite hand to actuate threading device to move twist 61 along filament 60. In another example, users or operators may use both hands to apply appropriate forces to expand and contract bifurcated arms cooperatively. Each individual user or operator could have their own timing with regard to expanding and contracting each threading device. Thus, the preceding example flows have been offered for purposes of teaching and discussion. Substantial flexibility is provided by the disclosed architecture in that any suitable arrangements, chronologies, configurations, and timing mechanisms may be provided without departing from the broad scope of this present disclosure.

Note also that the example embodiments described above can be replaced with a number of potential alternatives where appropriate. The processes and configurations discussed herein only offer some of the numerous potential applications of threading devices 10, 110, 110″ and 310. The elements and operations listed in FIGS. 1-21 may be achieved with the use of threading devices 10, 110, 110″, and 310, or any suitable variations thereof, in any number of contexts and applications. Accordingly, suitable infrastructure may be included within a given system or may cooperate with threading devices 10, 110, 110″, and 310 to effectuate the tasks and operations of the elements and activities associated with performing a threading procedure.

The various embodiments shown and described herein have been shown in a particular size relative to a human adult hand. It should be understood that each of the threading devices shown and described herein may be scaled to a smaller or larger size. For example, an overall size of the threading devices may be scaled down to minimize bulk and to possibly allow more control when placing filament 60 around hairs to be removed.

Although the present disclosure references particular embodiments in FIGS. 1-21, it should be understood that various other changes, substitutions, and alterations may be made hereto without departing from the sphere and scope of the present disclosure. For example, although the preceding FIGURES have referenced a number of components as participating in the outlined procedures, any suitable equipment or relevant tools may be readily substituted for such elements and, similarly, benefit from the teachings of the present disclosure. For example, certain users or operators may prefer to apply a contracting force to expand or open looped thread, or may prefer not having any spring mechanism but would rather apply all expanding and contracting forces with their own hands. Hence, the present tool may be designed based on particular criteria with particular scenarios envisioned.

It is also imperative to note that although the present disclosure implicates example procedures, this has only been done for purposes of example. Threading devices 10, 110, 110″, and 310 could readily be used in virtually any procedure where it would be beneficial and, accordingly, should be construed as such. Numerous other changes, substitutions, variations, alterations, and modifications may be ascertained to one skilled in the art and it is intended that the present disclosure encompass all such changes, substitutions, variations, alterations, and modifications as falling within the spirit and scope of the appended claims.

What is claimed is:

1. An apparatus, comprising:
an actuating body; and
first and second bifurcated arms each connected to the actuating body and each including a distal tip having an opening defined therein for slidably receiving a filament,
wherein the bifurcated arms are movable from a first position to a second position having a maximum distance defined between the distal tips when an opening force is applied to the actuating body.

2. The apparatus of claim 1, wherein the actuating body includes,
a first member and a second member; and
a pivoting mechanism coupling the first and second members together.

3. The apparatus of claim 2, wherein each of the bifurcated arms includes a proximal end attached to one of the first and second members.

4. The apparatus of claim 3, wherein the pivoting mechanism is an elastic sleeve surrounding the proximal ends of the pair of bifurcated arms.

5. The apparatus of claim 4, wherein the bifurcated arms are biased to the first position by the elastic sleeve.

6. The apparatus of claim 2, wherein a proximal end of the first arm is integral with the first member and a proximal end of the second arm is integral with the second member.

7. The apparatus of claim 1, wherein the opening force is a contracting force.

8. The apparatus of claim 1, wherein the bifurcated arms are movable from the second position to the first position when the opening force is released from the actuating body, the first position having a minimum distance defined between the distal tips.

9. The apparatus of claim 1, wherein each of the distal tips has a slit formed therein extending from an edge of the respective distal tip to the opening in the respective distal tip.

10. A method, comprising:
providing first and second devices, each of which includes an actuating body coupled to a pair of bifurcated arms, wherein a filament is secured by each distal tip of the bifurcated arms of the first and second devices;
moving at least one of the first and second devices to create tension in the filament;
forming a twist in the filament between the distal tips of the first pair of bifurcated arms and the distal tips of the second pair of bifurcated arms with the twist separating a first portion of the filament adjacent to the first device and a second portion of the filament adjacent to the second device;
placing the first portion of the filament around hairs to be removed from a body;
applying an opening force to the actuating body of the second device to move the bifurcated arms from a first position to a second position having a widened distance between the distal tips, wherein the twist travels along the filament toward the first device removing the hairs from the body.

11. The method according to claim 10, wherein the actuating bodies of the first and second devices each include pivotally connected first and second members.

12. The method according to claim 11, wherein the opening force is a contracting force applied to one end of the first and second members of the actuating body of the second device.

13. The method according to claim 10, wherein each of the distal tips of the bifurcated arms of the first and second devices includes an opening formed therein for slidably receiving the filament.

14. The method according to claim 13, wherein the filament is formed into a closed loop.

15. The method of claim 14 wherein each of the first and second portions of the filament is substantially triangular in shape when first and second devices are moved away from each other to create tension in the filament.

16. The method according to claim 10, further comprising:
applying the opening force to the actuating body of the first device before placing the first portion of the filament around the hairs to be removed, wherein the bifurcated arms of the first device are moved from the first position to the second position, whereby the first portion of the filament is enlarged and the twist is moved toward the second device.

17. The method of claim 16, further comprising:
releasing the opening force on the actuating body of the first device, wherein the bifurcated arms are moved from the second position toward the first position having a narrowed distance between the distal tips.

18. A threading system, comprising:
a first device including,
a first actuating body; and
a first pair of bifurcated arms coupled to the first actuating body, each of the first pair of bifurcated arms having distal tips adapted for securing a filament thereto; and
a second device including,
a second actuating body; and
a second pair of bifurcated arms coupled to the second actuating body, each of the second pair of bifurcated arms having distal tips adapted for securing the filament thereto;
wherein the first and second pairs of bifurcated arms are each movable from a first position to a second position having a maximum distance defined between the respective distal tips when an opening force is applied to the respective first and second actuating bodies.

19. The system of claim 18, wherein each of the distal tips of the first and second pairs of bifurcated arms include openings formed therein for slidably receiving a portion of the filament, wherein the filament is formed into a closed loop.

20. The threading system of claim 18, wherein each of the first and second actuating bodies includes,
first and second members; and
a pivoting mechanism coupling the first and second members together.
